(19) United States
(12) Patent Application Publication
(10) Pub. No.: US 2003/0195437 A1
(43) Pub. Date: Oct. 16, 2003

(54) Detecting and positioning device for tumor structures in subcutaneous tissue

(76) Inventor: Chiu-Chan Ho, Taipei City (TW)

Correspondence Address: Chiu-Chan Ho, 235 Chung - Ho, Box 8-24, Taipei, TW

(21) Appl. No.: 10/118,701

(22) Filed: Apr. 10, 2002

(51) Int. Cl. A61B 5/103

(52) U.S. Cl. 600/587

(57) ABSTRACT

A detecting and positioning device for tumor structures in a subcutaneous tissue is disclosed. The device has a bag body with an inlet. One side of the bag body having a cover set. The bag body is filled with liquid; and the inlet is sealed by high frequency supersonic sticking way. In operation, hands of a user inserts into the cover set for operating the bag body. The cover set is formed by a cambered cover set and two cambered cover units. The cambered cover set has a plurality of sub-cambered cover units. Moreover, in another application of the present invention, the cover set has only one chamber with an opening, thereby, one hand of the user inserts into the cover set from the opening.

### FIELD OF THE INVENTION

The present invention relates to devices for checking breast cancer, and particularly to a detecting and positioning device for tumor structures in a subcutaneous tissue, which can resolve the problems induced in the prior art. The operation of the device is easy. The user can operate it by left or right hand. Small tumor structures in the subcutaneous tissue can be detected with a higher sensitivity.

### BACKGROUND OF THE INVENTION

Recently, breast cancer has been a very important factor about the death of women. More and more young women catch breast cancer. However, if the breast cancer can be detected early, it has a larger possibility to be healed. Unluckily, in many areas, women have a sense of shame for being checked by a doctor. Therefore, it is often that the time for curing breast cancer is delayed and the women must face the threat of breast cancer. Moreover, it is time-consumed to be checked by doctors, and thus many women do not accept the check of breast cancer.

A better way for resolving above mentioned problem is that the check work is performed by women themselves. Conventionally, people determine whether they have catches breast cancer by using a hand to touch the breast for finding tumor structure in the subcutaneous tissue. The fingers must move along spiral paths continuously. Due to the friction force between the subcutaneous tissue and fingers, the sensitivity is very low and thus smaller tumor structure can not be found. Therefore, it is often that the breast cancer can not be found in the initial stage. Indeed, this prior art way is not ideal.

### SUMMARY OF THE INVENTION

Accordingly, the primary object of the present invention is to provide a detecting and positioning device for tumor structures in a subcutaneous tissue, which can resolve the problems induced in the prior art. The operation is easy. The user may operate it by left or right hand. Small tumor structures in the subcutaneous tissue can be detected with a higher sensitivity.

To achieve above-mentioned problem, the present invention provides a detecting and positioning device for tumor structures. The device has a bag body with an inlet. One side of the bag body has a cover set. The bag body is filled with liquid; and the inlet is sealed by high frequency supersonic sticking way. In operation, hands of a user inserts into the cover set for handling the bag body.

The cover set is formed by a cambered cover set and two cambered cover units. The cambered cover set has a plurality of cambered cover units.

In another application of the present invention, the cover set has only one chamber with an opening, thereby, one hand of the user inserts into the cover set from the opening.

The various objects and advantages of the present invention will be more readily understood from the following detailed description when read in conjunction with the appended drawing.

### BRIEF DESCRIPTION OF THE DRAWINGS

FIG. 1 is an exploded view of the first embodiment of the present invention.

FIG. 2 is an assembled view of the second embodiment of the present invention.

FIG. 3 shows one application of first embodiment of the present invention.

FIG. 4 shows an appearance of the second embodiment of the present invention.

FIG. 5 is a partial cross section view of the second embodiment of the present invention.

FIG. 6 shows one appearance of the third embodiment of the present invention.

FIG. 7 is a partial cross section view of the third embodiment of the present invention.

### DETAILED DESCRIPTION OF THE PREFERRED EMBODIMENTS

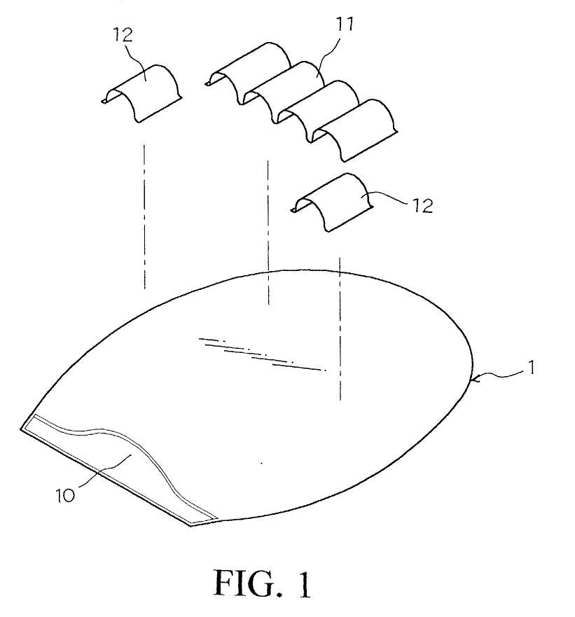

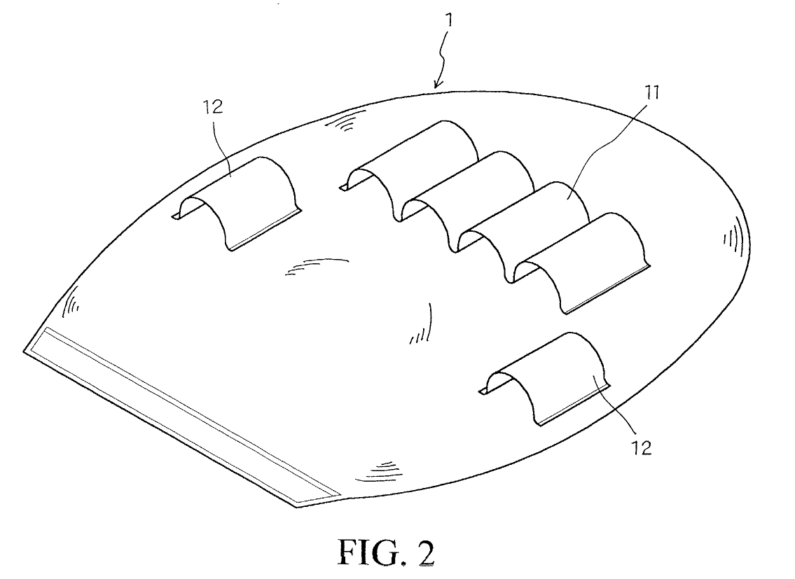

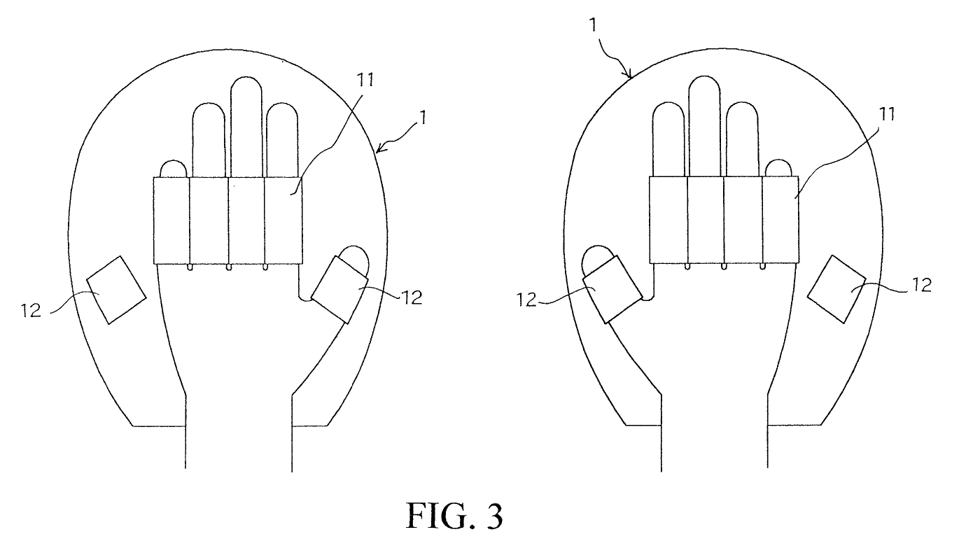

Referring to FIGS. 1 to 3, the detecting and positioning device for tumor structures in a subcutaneous tissue of the present invention is illustrated. The detecting and positioning device for tumor structures in a subcutaneous tissue of the present invention has a hollow bag body 1. The bag body 1 may be integrally formed or be formed by two pieces the edges of which are seamed by high frequency supersonic technology. The edge of the bag body 1 is sealed and liquid inlet 10 is formed at one edge. One surface of the bag body 1 is stuck with a cambered cover set 11 having a plurality of cambered cover units. The cambered cover set 11 is stuck by high frequency supersonic technology. The left and right sides of the cambered cover set 11 have respective cambered cover units 12. In this embodiment, each of the cambered cover units in the cambered cover set 11 and cambered cover units 12 has two opened ends. Thereby, the fingers of right or left hands can be inserted into the cambered cover set 11 and one of the cambered cover units 12, as illustrated in FIG. 3. Thus, the bag body 1 can be operated conveniently and easily. Liquid, such as oil liquid or lubricating oil can be filled into the bag body 1 through the inlet 10. Then the opening of the inlet 10 is sealed by high frequency supersonic technology. As a result, a detecting and positioning device for tumor structures in a subcutaneous tissue having a bag body 1 with a cambered cover set 11 and cambered cover units 12 is constructed, as illustrated in FIG. 2.

The operation of the present invention will be described herein with referring to FIG. 3. The user can wear the bag body 1 by passing the fingers through the cambered cover set 11 and one cambered cover unit 12. Next, another surface of the bag body 1 without the cambered cover set 11 touches one bosom. Thereby, the finger applied a force upon the bag body 1 will contact indirectly the bosom through the liquid in the bag body 1. Thereby, no larger friction occurs. The liquid in the bag body 1 presents a lubricating effect. The friction force sensed by the fingers is reduced and thus the sensitivity is increased. This is beneficial for detecting an unobvious and needle-like tumor structure. When the bag body 1 covers the skin and the fingers touch a slight protrusion or an unobvious tumor structure, the liquid in the bag body 1 will amplify the touching effect so that the user can identify the tumor structure easily with a high sensitivity and high accuracy. Moreover, the tumor structure of the subcutaneous tissue can be detected early and the treatment can be performed.

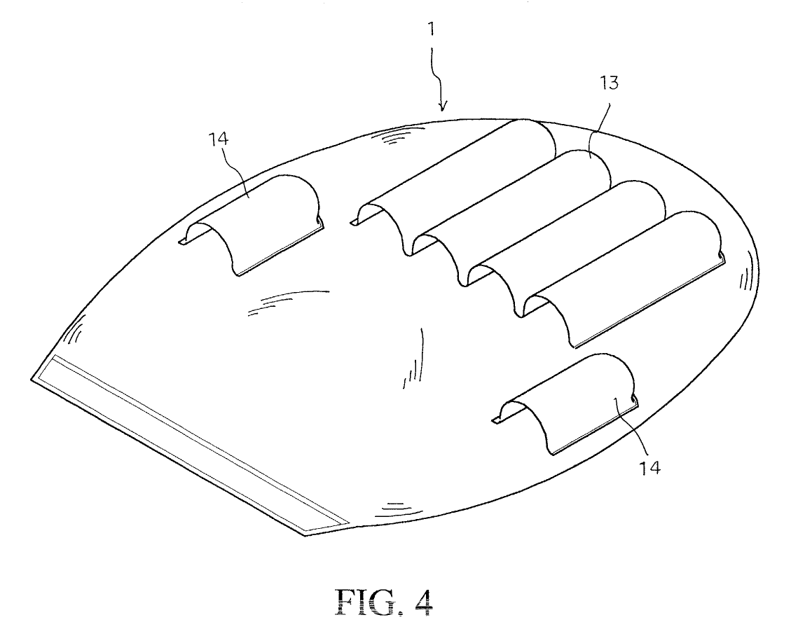

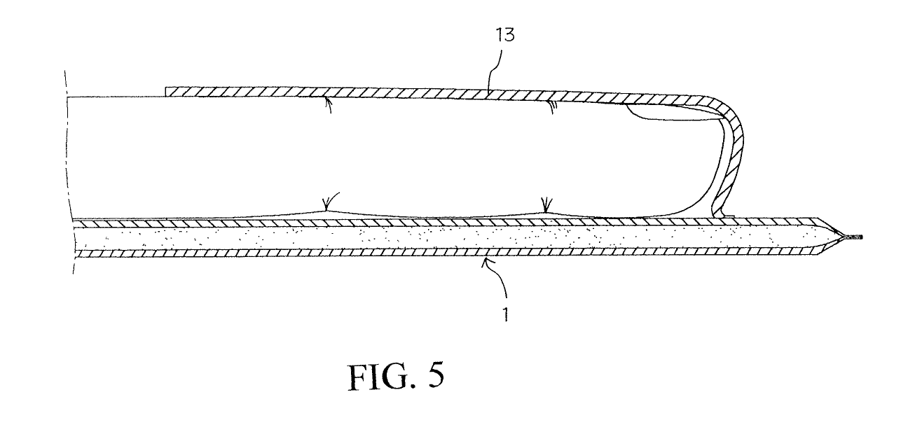

Referring to FIGS. 4 and 5, another embodiment of the present invention is illustrated and a partial cross section view of the present invention is shown. It is illustrated that instead of using the integral formed cambered cover set 11 and cambered cover units 12 which are opened at two ends, in this embodiment, a cambered cover set 13 and two cambered cover units 14 are used. The cambered cover set 13 has a plurality of cambered cover units. Each of the cambered cover units in the cambered cover set 13 and the two cambered cover units 14 has one end which is closed. Thereby, when fingers insert into the cambered cover units, the tips of the fingers are enclosed in the cambered cover units.

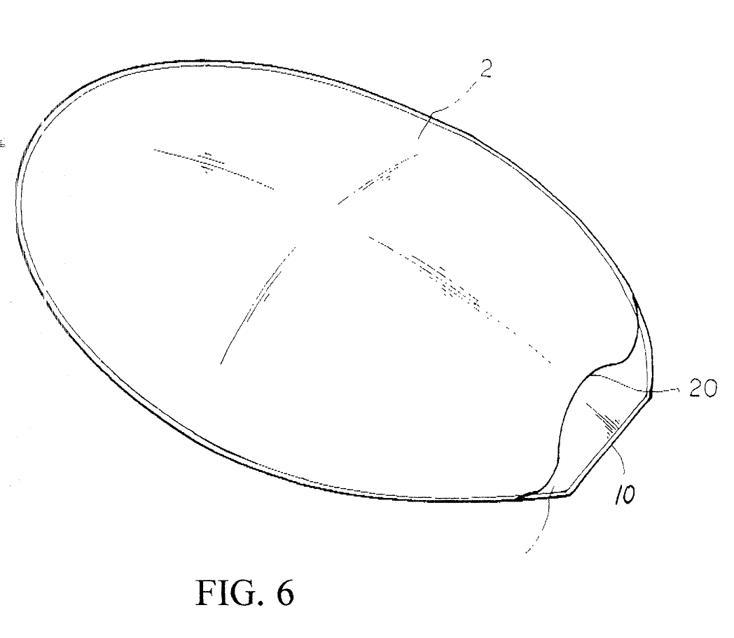

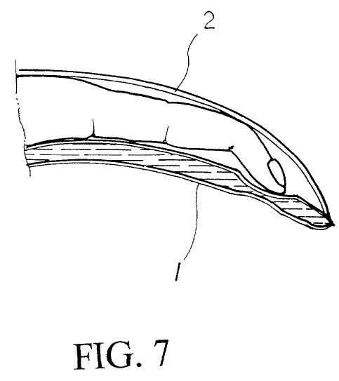

With reference to FIGS. 6 and 7, another embodiment of the present invention is illustrated. In this embodiment, the cambered cover set 11 and cambered cover units 12 in the first embodiment is replaced by a palm cover 2. One lateral side of the bag body 1 is installed as a concave opening 20. The hand of the user may insert into the palm cover from the concave opening 20.

In summary, in the embodiments of the present invention, a cambered cover set 11 and two cambered cover unit 12 which are opened at two ends or at only one ends, or a palm cover is stuck on one surface of the bag body 1. Thereby, the user's fingers or hand can be placed in the cover and thus the user can operate the bag body 1 easily and conveniently. The design of the present invention can be used for a right or left hand.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A detecting and positioning device for tumor structures in a subcutaneous tissue, the detecting and positioning device having a bag body with an inlet; one side of the bag body having a cover set; the bag body being filled with liquid; and the inlet being sealed by high frequency supersonic sticking way, wherein in operation, hands of a user inserts into the cover set for operating the bag body.

2. The detecting and positioning device for tumor structures in a subcutaneous tissue as claim in claim 1, wherein the cover set is formed by a cambered cover set and two cambered cover units; the cambered cover set has a plurality of sub-cambered cover units; in using, the thumb of the right hand or left hand of the user is inserted into one of the cambered cover unit and the other four fingers of the respective hand inserts into the sub-cambered cover unit of the cambered cover set.

3. The detecting and positioning device for tumor structures in a subcutaneous tissue as claim in claim 1, wherein the cover set has only one chamber with an opening, thereby, one hand of the user inserts into the cover set from the opening.

4. The detecting and positioning device for tumor structures in a subcutaneous tissue as claim in claim 1, wherein the bag body is a one piece bag body.

5. The detecting and positioning device for tumor structures in a subcutaneous tissue as claim in claim 1, wherein the bag body is a formed by two pieces which are sealed by high frequency supersonic sticking way.

6. The detecting and positioning device for tumor structures in a subcutaneous tissue as claim in claim 1, wherein the liquid in the bag body is one of a group selected from lubricating oils and harmless oils.
